(19) United States
(12) Patent Application Publication
(10) Pub. No.: US 2026/0369360 A1
(43) Pub. Date: Nov. 19, 2026

(54) PCR SYSTEM AND METHOD FOR AUTO-THRESHOLDING

(71) Applicant: Life Technologies Corporation, Carlsbad, CA (US)

(72) Inventors: Sitong Liu, Santa Clara, CA (US); Nivedita Sumi Majumdar, Martinez, CA (US); Roger Dettloff, Palo Alto, CA (US); Yun Zhu, San Diego, CA (US)

(21) Appl. No.: 19/475,846

(22) PCT Filed: Apr. 17, 2024
(86) PCT No.: PCT/US2024/025021

§ 371 Date: Oct. 16, 2025

Related U.S. Application Data
(60) Provisional application No. 63/460,882, filed on Apr. 20, 2023.

(51) Int. Cl. G16B 40/10; G16B 25/20; G16B 40/30

(52) U.S. Cl. CPC G16B 40/10; G16B 25/20; G16B 40/30

(57) ABSTRACT

Embodiments of a digital polymerase chain reaction (dPCR) system and method having improved auto-thresholding performance and accuracy are disclosed. One embodiment of the disclosure comprises a dPCR processing module including an au-to-thresholding agent that dynamically analyzes and selects between Gaussian Mixture Modeling (GMM) and K-means clustering for use in auto-thresholding results of a dPCR assay using a dPCR instrument to improve dPCR measurement technology.

### CROSS-REFERENCE TO RELATED APPLICATIONS

This application claims the benefit of U.S. Provisional Application No. 63/460,882 filed Apr. 20, 2023. This application also has some subject matter relationship to commonly assigned Provisional application numbers: U.S. 63/460,884; U.S. 63/460,879, and U.S. 63/460,885, all filed on Apr. 20, 2023. The contents of these applications are incorporated herein by reference in their entirety.

### BACKGROUND

This disclosure relates generally to image processing systems and methods for measuring (e.g., quantifying) presence of a target analyte in a sample using digital polymerase chain reaction (dPCR).

dPCR can be performed on biological samples that contain or are suspected to contain a target analyte of interest, such as a cell, tissue, or specimen such as hair, a biological fluid such as blood, urine, saliva, etc., a cell cluster such as a microbial colony, or an organism, cell, microbe, bacterium, virus, protein, antibody, or nucleic acids such as such as DNA or RNA molecules. Target analytes include “original” analytes that were originally present in the biological sample as well any “synthetic” analytes that are indicative of the presence of original analytes which may be added or generated during detection, including PCR amplicons, antigen-antibody complexes, etc. Digital quantification (e.g., digital PCR) begins with a sample including a relatively small number of a target analyte, e.g., a polynucleotide or nucleotide sequence template DNA (or RNA). The sample is partitioned into a large number of smaller test samples, which will ideally contain either one (or a small number close to one) target analyte or none of the target analytes such that a separate detection reaction can be carried out in each partition individually. Suitable partitions allow individual targets to be sufficiently distanced from other individual targets to allow for individual detection or quantification. Partitions may or may not include separating barriers such as walls or membranes or liquids that are immiscible with the sample, or semisolid media. Exemplary partitions include individually distanced targets, e.g., deposited on a substrate such as a glass slide, a tube, open or closed well, droplet, vesicle, chamber or bead, or any representation of an individual signal derived from a target that is distinguishable over background or noise, for example a bright spot over a darker background in a digital or analog image. In digital PCR methods, when the samples are thermally cycled using a PCR apparatus, the samples containing the target are amplified and produce a positive detection signal, while the samples that do not contain the target are not amplified and produce no detection signal. After multiple PCR amplification cycles, the samples are imaged and analyzed for fluorescence, which is used to quantify the target concentration in the samples.

Typically, dPCR involves partitioning a PCR solution that includes the sample into tens of thousands of nano-liter sized droplets, where a separate PCR reaction takes place in each one. The PCR solution may include, for example, template DNA (or RNA), fluorescence-quencher probes, primers, and a PCR master mix, which contains DNA polymerase, dNTPs, MgCl₂, and reaction buffers at optimal concentrations. Several different methods can be used to partition samples, including microwell plates, capillaries, oil emulsion, and arrays of miniaturized chambers with nucleic acid binding surfaces. This partitioning of samples allows a more reliable collection and sensitive measurement of nucleic acid amounts. The method has been demonstrated as useful for studying variations in gene sequences, such as copy number variants and point mutations, and it is routinely used for clonal amplification of samples for next-generation sequencing.

Typically, dPCR instrumentation includes a microplate having a large number of partitions (e.g., over 1,000, over 20,000, over 100,000, etc.). A fluorescent dye functions as an intercalating agent that emits detectable fluorescence when bound to DNA (e.g., human DNA or DNA of an infectious agent). As the PCR progresses and the quantity of DNA increases, more dye binds to the PCR products and hence, signal intensity increases. The dPCR instrumentation also includes a thermal cycler for amplification, and a light source for excitation of fluorescent probes associated with the plurality of partitions. A camera captures the fluorescent reactions of the plurality of partitions and deliver the image to a processor-based device enabled to control the instrument, detect fluorescent datasets, and process data for further analysis. Specifically, after a PCR amplification cycle number designated to be an “endpoint” cycle, the sample partitions are checked for fluorescence, and, based on comparison to a threshold level, a fluorescence value for a given sample partition is considered to represent presence (amplification) or absence (no amplification) of a target analyte. Determining an appropriate threshold level is important to obtaining accurate results from dPCR technology.

### SUMMARY

Given that many thousands, or even hundreds of thousands of separate PCR reactions and corresponding fluorescence measurements typically result from a dPCR assay, efficient computerized processing of the resulting data is essential to allow dPCR to be a useful technology. Computerized auto-thresholding of dPCR data can enhance dPCR throughput and accuracy and therefore improves the efficiency and performance of dPCR instruments.

One approach to auto-thresholding relies on automated cluster analysis. Cluster analysis can be used to identify an appropriate threshold for either distinguishing between two (or more) clusters of data points corresponding to underlying populations or determining whether a particular data point is part of a single cluster corresponding to an underlying population.

Typically, a particular cluster analysis approach is selected that is best suited for a given application. However, embodiments of the present disclosure improve dPCR technology by implementing computerized auto-thresholding that intelligently selects between different clustering algorithms based on resolution determinations associated with cluster analysis of a given population of dPCR fluorescence measurements. In some embodiments, a thresholding formula associated with results from the selected clustering algorithm is used to set an auto-threshold for distinguishing between fluorescence values representing amplification for given partitions and those representing non-amplification.

Gaussian Mixture Modeling (GMM) (e.g., sklearn.mixture Gaussian Mixture Python Library) is one useful approach to automated cluster analysis that can be applied to dPCR data. A Gaussian mixture model is a probabilistic model that assumes all the data points are generated from a mixture of a finite number of Gaussian distributions with unknown parameters. If two clusters are assumed, GMM can fit two Gaussian distributions to the dPCR data, assign probabilities of each point belonging to a given cluster, and can return a mean and standard deviation for each cluster. The mean and standard deviation of each cluster can be used to compute a threshold value distinguishing between the two clusters.

However, GMM does not always identify two clusters in dPCR data with sufficiently high resolution. In such instances, one cluster (rather than two) can be assumed and a probability can be assigned to each data point defining how likely it is that the datapoint can be assumed to belong to the cluster. For some dPCR data sets, this might be an appropriate assumption if, for example, the target analyte was completely absent from the sample and none of the sample partitions had amplification results consistent with presence of the target. Alternatively, the two-cluster assumption can be maintained, but, in that case, the risk of errors in using GMM for setting a threshold to assign binary classification might be unacceptably high for many of the datapoints.

Embodiments of the present disclosure recognize that, although GMM provides the best results in many cases, for some dPCR data sets, other clustering algorithms or mathematical models, such as, for example, K-means clustering, can better distinguish between two clusters. Therefore, dynamically selecting between two clustering models as part of auto-threshold setting can increase the accuracy of dPCR technology. With this in mind, some embodiments of the present disclosure dynamically select between different clustering models for use in auto-thresholding dPRC results. In some embodiments, resolution analysis is used to dynamically determine whether to use GMM clustering for threshold setting or whether to use K-means clustering.

It should be appreciated that embodiments of the present disclosure can be implemented in numerous ways, such as a process, an apparatus, a system, a device, an article of manufacture, or a method. Details of several embodiments are further described below. Further details of embodiments of systems and methods consistent with the present disclosure are disclosed herein.

### BRIEF DESCRIPTION OF THE DRAWINGS

The present application can be best understood by reference to the embodiments described below taken in conjunction with the accompanying drawing FIGURES, in which like parts may be referred to by like numerals.

FIG. 1A illustrates a networked dPCR system having an auto-thresholding agent with improved performance in accordance with an embodiment of the present disclosure.

FIG. 1B is a block diagram of the auto-thresholding agent of the server of FIG. 1, in accordance with some embodiments.

FIG. 2A is a flow diagram of a method for auto-thresholding of dPCR operative system of FIG. 1, in accordance with some embodiments.

FIG. 2B is a flow diagram of a method for GMM fit as applied to the site statistics data of the auto-thresholding method of FIG. 2A, in accordance with some embodiments.

FIG. 2C is a flow diagram of a method for K-MEANS fit as applied to the site statistics data of the auto-thresholding method of FIG. 2A, in accordance with some embodiments.

FIG. 2D is a flow diagram of a method for calculating one population threshold using GMM of the auto-thresholding method of FIG. 2B, in accordance with some embodiments.

FIG. 2E is a flow diagram of a method for calculating two population threshold using GMM of the auto-thresholding method of FIG. 2B, in accordance with some embodiments.

FIG. 2F is a flow diagram of a method for calculating two population threshold using K-means of the auto-thresholding method of FIG. 2C, in accordance with some embodiments.

FIG. 3 displays an illustration showing an example of a computing device which may implement the examples described herein.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

In some embodiments, the dPCR operative system and method of processing datasets associated with biological sampling distributed across an array of microchambers of a microplate includes a processor-based automatic thresholding (auto-thresholding) agent, having improved auto-thresholding performance and increased accuracy of threshold fluorescent dye setting. Particularly, this dPCR operative system having improved auto-thresholding performance and accuracy is comprised of dPCR processing module including an auto-thresholding agent that uses both Gaussian Mixture Modeling (GMM) and K-means clustering. The auto-thresholding method includes determining whether a one or two population cluster of data points exist within the associated fluorescent data corresponding to a plurality of optical signals detected at a dPCR instrument. In one example, the method includes detecting whether two clusters exist of two distinct populations using GMM. In response to detecting two clusters, the two-population GMM threshold is assigned as the final threshold. In absence of detecting two distinct populations using GMM, the method detects the existence of two clusters using K-means clustering. When two clusters are detected, the two-population K-means clustering threshold (K-means threshold) is assigned as the final threshold. When two clusters are not detected, the one-population GMM threshold is assigned as the final threshold.

In one example, the dPCR operative system having improved auto-thresholding performance and accuracy is comprised of a server coupled to a dPCR instrument having at least one microplate. The server includes at least one processor coupled to at least one memory and a dPCR processing module including an auto-thresholding agent. The auto-thresholding agent a cluster detection unit having a receiver, a Gaussian Mixture Modeling (GMM) engine, and a K-means clustering engine. The receiver coupled to the at least one memory having computer-readable instructions stored thereon that, when executed by the at least one processor cause the at least one processor to receive, from a dPCR instrument, fluorescent data corresponding to a plurality of optical signals detected at a dPCR instrument. The fluorescent data includes a plurality of individual points associated with a plurality of microchambers, where the plurality of individual points form one or more clusters sharing a relative property. The GMM engine couples to the receiver and the at least one memory having instructions that cause the at least one processor to detect whether the one or more clusters are two clusters associated with two distinct populations using GMM. The GMM engine in cooperation with the processor is also operable to assign, in response to detecting two clusters using GMM, a two-population GMM threshold to be a final threshold. In absence of detecting two distinct populations using GMM, the K-means clustering engine in cooperation with the processor is operable to detect whether the number of the one or more clusters is two clusters associated with a two-cluster population using K-means clustering. In response to detecting two clusters using K-means clustering, the K-means clustering engine in cooperation with the processor is operable to assign a two-population K-means clustering threshold to be the final threshold. In response to absence of detecting two clusters using K-means clustering, the K-means clustering engine in cooperation with the processor is operable to assign a one-population GMM threshold to be the final threshold.

Other aspects and advantages of the embodiments will become apparent from the following detailed description taken in conjunction with the accompanying drawings which illustrate, by way of example, the principles of the described embodiments.

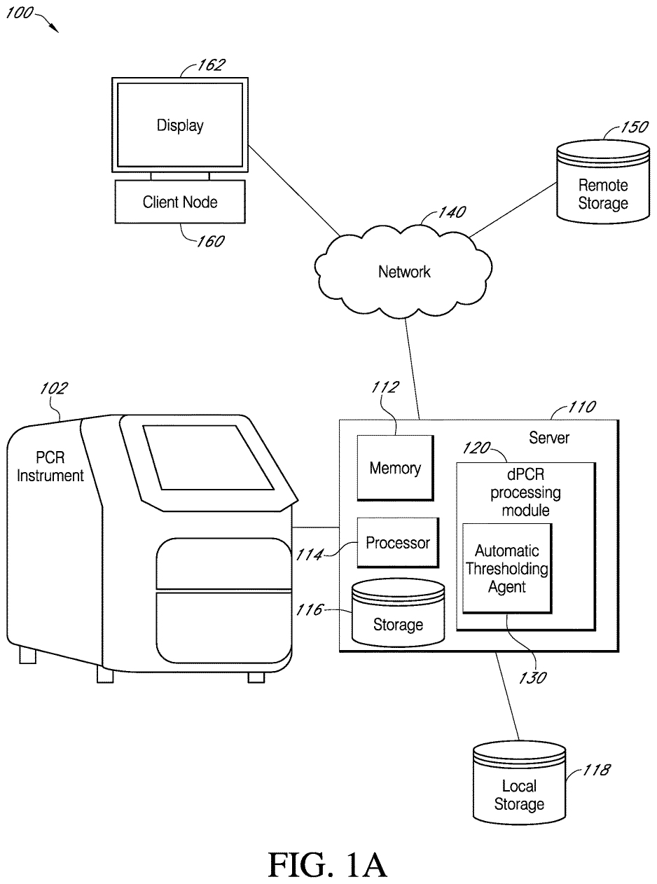

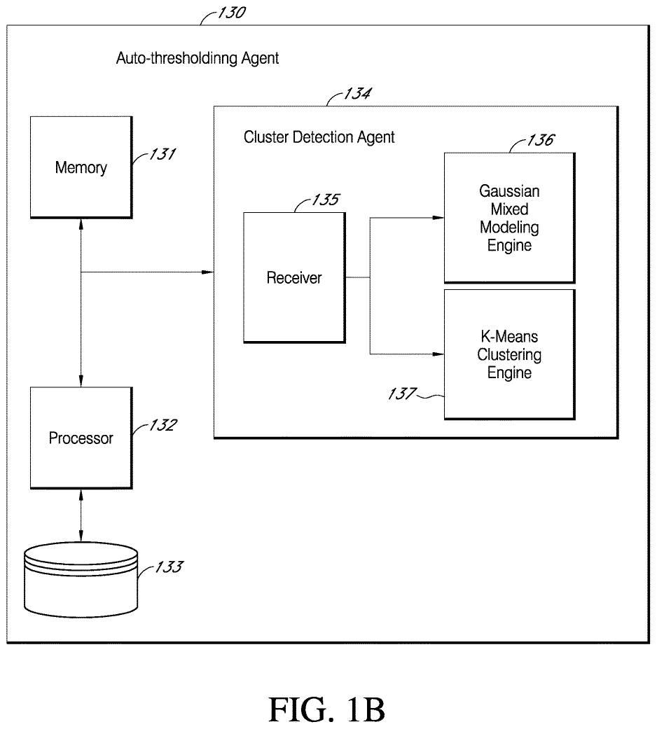

FIG. 1A illustrates networked digital Polymerase Chain Reaction (“dPCR”) system 100 having an auto-thresholding agent 130 with improved performance in accordance with an embodiment of the present disclosure. System 100 comprises dPCR Instrument 102, at least one client node 160, and server 110. Digital PCR instrument 102 includes microplate (not shown) having an array of microchambers filled with a biological sample. Instructions for implementing dPCR reside in auto-thresholding agent 130 (a computer program product) within dPCR processing module 120 which is stored in storage 116 and those instructions are executable by processor 114. When processor 114 is executing the instructions of dPCR processing module 120, the instructions, or a portion thereof, are typically loaded into working memory 112 from which the instructions are readily accessed by processor 114. In the illustrated embodiment, auto-thresholding agent 130 is stored in local storage device 118 or another non-transitory computer readable medium (which may include being distributed across media on different devices and different locations).

Although not shown, dPCR instrument 102 includes a microplate (not shown) having the plurality of partitions including a fluorescent dye solution. The dye functions as an intercalating agent that emits detectable fluorescence when bound to double-stranded DNA or infectious agent. As the PCR progresses and the quantity of double-stranded DNA increases, more dye binds to the PCR products and hence, signal intensity increases. Digital PCR instrument 102 also includes a thermal cycler (not shown) for amplification, and a light source (not shown) for excitation of fluorescent probes associated with the plurality of partitions. A camera (not shown) couples to record the fluorescent reactions of the plurality of partitions and deliver the image to dPCR processing module 120 enabled to control the instrument, detect fluorescent datasets, and perform auto-thresholding. Specifically, after multiple PCR amplification cycles, the samples are checked for fluorescence, where dPCR processing module 120 detects a binary readout of “0” or “1” associated with the array of microchambers.

In one embodiment, processor 114 comprises multiple processors, which may comprise additional working memories (additional processors and memories not individually illustrated) including a graphics processing unit (GPU) comprising at least thousands of arithmetic logic units supporting parallel computations on a large scale. GPUs are often utilized in deep learning applications because they can perform the relevant processing tasks more efficiently than can typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application-specific), field programmable gate arrays and the like, or combinations thereof. In some embodiments, however, a processor such as processor 114 may be implemented as one or more general purpose processors (preferably having multiple cores) without necessarily departing from the spirit and scope of the present invention.

Client node 160 includes display 162 for displaying results of processing carried out by dPCR processing module 120. In alternative embodiments, auto-thresholding agent 130, or a portion thereof, may be stored in storage devices and executed by one or more processors residing on PCR instrument 102 and/or client node 160. Such alternatives do not depart from the scope of the invention.

In some embodiments, system 100 includes a network 140 coupled between the server 110 and client node 160. Although not shown, in some embodiments server 110 and client node 160 couple directly to one another without network 140. The system may also include at least one dumb terminal (not shown), such as a land line, cell phone, pager monitor, and the like. Although not shown, server 110 may also be coupled to a conventional telephone (not shown) by Public Switched Telephone Network (PSTN), which couples to the network 110. The server 110 may couple to the remote storage device 150 for reference to prior PCR analysis results and versions of other parameters described below. In some embodiments, each client node 160 includes dPCR/qPCR agent 104, data analysis system 102, memory (not shown), a processor (not shown), and local data store (not shown).

In some embodiments, server 110, having dPCR processing module 120 and auto-thresholding agent 130, communicates with each client node 160 and serves as the sole agent that performs the method of PCR described herein. In other embodiments, a copy of the auto-thresholding agent (not shown) within client node 160 serves as a device that communicates with the server 110 to perform the method of auto-thresholding in real-time described more in detail below. The one or more client nodes 160, server 110, and the remote storage device 150 may reside on the same LAN, or on different LANs that may be coupled together through the Internet, but separated by firewalls, routers, and/or other network devices. In one embodiment, one or more client nodes 160 couple to network 110 through a mobile communication network. In another embodiment, one or more client nodes 160, server 110, and the remote storage device 150 reside on different networks. In some embodiments, server 110 resides in a cloud network. Although not shown, in various embodiments, one or more client nodes 160 may be notebook computers, desktop computers, microprocessor-based or programmable consumer electronics, network appliances, mobile telephones, smart telephones, pagers, radio frequency (RF) devices, infrared (IR) devices, Personal Digital Assistants (PDAs), set-top boxes, cameras, integrated devices combining at least two of the preceding devices, and the like. FIG. 1A illustrates that dPCR processing module 120 may also entirely or partially operate PCR instrument 102. The one or more client nodes 160 may include a PDA, a Global Positioning System (GPS) device, a monitor, an interactive television, and Internet protocol (IP) phone, a pager, a cellular phone, a satellite phone, and the like.

In some embodiments, the one or more client nodes 160 comprise a processor (not shown), memory (not shown), and a copy of auto-thresholding agent 130. In some embodiments, the one or more client nodes 160 may comprise processing software instructions and/or hardware logic required for PCR auto-thresholding analysis according to the embodiments described herein.

In some examples, server 110 provides remote cloud storage capabilities for PCR analysis and various types of PCR policies associated, through the remote storage device 150 coupled by network 140. Additionally, server 110 provides remote storage capabilities for PCR analysis data. In some embodiments, server 110 retrieves previous results relating to PCR analysis data and policies relating to the same from a remote datastore 150 to a local data store 118. In other embodiments, the database of PCR auto-thresholding policies, prior detection results, and the like may be stored locally on the one or more client nodes 160, local storage 116, or the server 110. In particular, for remote storage purposes, the local data storage unit 118 can be one or more centralized data repositories having mappings of respective associations between each fragment data and its location within remote storage devices 150. Local data store 116 may represent a single or multiple data structures (databases, repositories, files, etc.) residing on one or more mass storage devices, such as magnetic or optical storage-based disks, tapes or hard drives. This local data store 116 may be an internal component of server 110. In the alternative, the local data store 118 couples externally to server 110, or remotely through a second network (not shown). Further, server 110 may communicate with remote storage devices over a public or private network. Although not shown, in various embodiments, the server 110 may be a notebook computer, desktop computer, microprocessor-based or programmable consumer electronics, network appliance, mobile telephone, smart telephone, radio frequency (RF) device, infrared (IR) device, Personal Digital Assistant (PDA), set-top box, an integrated device combining at least two of the preceding devices, and the like.

FIG. 1B is a block diagram of the auto-thresholding agent 130 of the server 110 of FIG. 1, in accordance with some embodiments. The auto-thresholding agent 130 comprises a cluster detection agent 134, memory 131, processor 132, and storage device 133. Cluster detection agent 134 includes a receiver 135, a GMM engine 136, and K-means clustering engine 137. Cluster detection agent 134 in cooperation with processor 132 couples to memory 131 and storage device 133 to retrieve and process auto-thresholding instructions. These auto-thresholding instructions can be stored in storage device 133. Receiver 135 couples to receive fluorescent data corresponding to a plurality of optical signals detected at dPCR instrument 102 (FIG. 1A), wherein the fluorescent data includes a plurality of individual points associated with a plurality of microchambers. Receiver 135 couples to both GMM engine 136 and K-means clustering engine 137 to deliver fluorescent data for detection of the existence of one or more populations and to generate a threshold, based upon the detected population.

In operation, a user of the dPCR system 100, having improved auto-thresholding performance and accuracy, opens a front panel to insert the microplate (not shown) within the dPCR instrument 102. Prior to placement, the user fills the array of microchambers of the microplate with a biological sample for dPCR analysis. Receiver 135 directs the at least one memory 131 of auto-thresholding agent 130 (having computer-readable instructions stored thereon that, when executed by the at least one processor 132) to enable receiver 135 to receive, from a dPCR instrument 102, fluorescent data corresponding to a plurality of optical signals detected at a dPCR instrument. The fluorescent data includes a plurality of individual points associated with a plurality of microchambers, where the plurality of individual points form one or more clusters sharing a relative property. GMM engine 136 couples to the receiver 135 and the at least one memory 131 having instructions that cause the at least one processor 132 to detect whether the fluorescent data (e.g., the one or more clusters) are associated with two distinct populations using GMM. In particular, GMM engine 136 detects the clusters of the fluorescent data by computing the mean and standard deviation associated with the GMM lower and GMM higher populations. GMM engine 136 calculates the threshold for one population and two populations, along with an effective resolution using the following:

$${GMM}_{{th}_{lo}} = ml + sl \qquad {GMM}_{{th}_{hi}} = mh - sh$$

$$GMM two population threshold = ( {GMM}_{{th}_{lo}} + {GMM}_{{th}_{hi}} ) / 2$$

$$If mean - SD > 2900. , GMM one population threshold = mean - 4 * SD$$

$$If mean - SD < 2900. , GMM one population threshold = mean + 4 * SD$$

$$GMM resolution = \frac{abs ( ml - mh )}{2 * ( sl + sh )}$$

where GMMₜₕₗₒ is the threshold for the GMM lower population, ml is mean of the GMM lower population, sl is standard deviation of the GMM lower population; GMMₜₕₕᵢ is the threshold for the GMM higher population, mh is mean of the GMM higher population, sh is standard deviation of the GMM higher population; SD is standard deviation of the whole population (dataset), GMMᵣₑₛₒₗᵤₜᵢₒₙ is the GMM resolution, abs means absolute value.

In response to detecting two clusters using GMM, GMM engine 136 in cooperation with the processor 132 assigns a two-population GMM threshold to be a final threshold. In one example, if the resolution from the GMM algorithm is greater than threshold1=0.7, then the GMM engine 136 determines that two populations exist, and uses the GMM two-population threshold as the final threshold. In absence of detecting two distinct populations using GMM, K-means clustering engine 137 in cooperation with processor 132 detects whether the number of the one or more clusters is two clusters associated with a two-cluster population using K-means clustering. In particular, K-means clustering engine 137 detects the clusters of the fluorescent data by computing the mean and standard deviation associated with the lower and higher populations. K-means clustering engine 137 calculates the threshold for one population and two populations, along with an effective resolution using the following:

$${Kmeans}_{{th}_{lo}} = ml + sl \qquad {Kmeans}_{{th}_{hi}} = mh - sh$$

$$Kmeans two population threshold = ( {Kmeans}_{{th}_{lo}} + {Kmeans}_{{th}_{hi}} ) / 2$$

$$Kmeans resolution = \frac{abs ( ml - mh )}{2 * ( sl + sh )}$$

where Kmeansₜₕₗₒ is the threshold for the K-means lower population, ml is mean of the K-means lower population, sl is standard deviation of K-means lower population; Kmeansₜₕₕᵢ is the threshold for the K-means higher population, mh is mean of K-means higher population, sh is standard deviation of the K-means higher population; SD is standard deviation of the whole population (data set), abs means absolute value.

In response to detecting two clusters using K-means clustering, K-means clustering engine 137 assigns a two-population K-means clustering threshold to be the final threshold. In one example, when the K-Means resolution is greater than 1.5, then K-means clustering engine 137 sets the threshold based on K-Means two population thresholding. In response to absence of detecting two clusters using K-means clustering, K-means clustering engine 137 assigns the one-population GMM threshold to be the final threshold.

In some embodiments, server 110 retrieves prior dPCR analysis results and auto-thresholding data from local storage units 116 and/or 118. In other embodiments, server 110 prior dPCR analysis results and versions of other parameters from remote storage 150. After performing auto-thresholding using the dPCR processing module 120, server 110 sends the resultant data of the dPCR analysis to client node 160. Display 108 of client node 160 displays this resultant data of the PCR analysis in graphical, textual, and various other formats, enabling user review and further analysis.

As note supra in reference to one example, the GMM derived resolution (res1) must be greater than a threshold of 0.7, in order for the GMM engine 136 to determine that two populations exist. In some examples, the K-means clustering derived resolution (res2) must be greater than a threshold of 1.5, in order for the K-means clustering engine 137 to determine that two populations exist. In another example, the standard deviation of the whole population derived using GMM must be greater than a maximum negative threshold of 2900.00, in order for the one population to be detected and an associated GMM threshold to be calculated. Such alternatives do not depart from the scope of the invention.

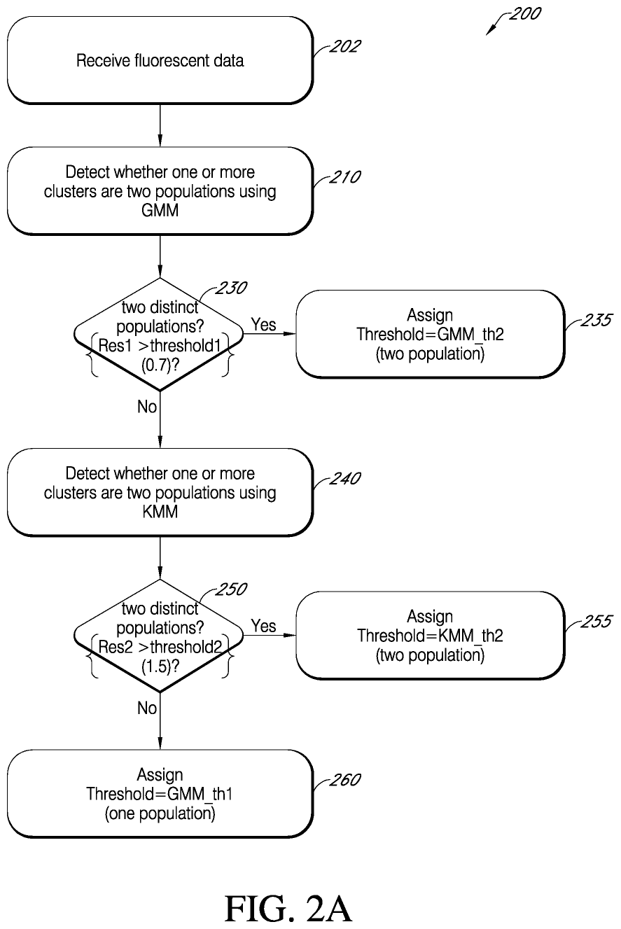

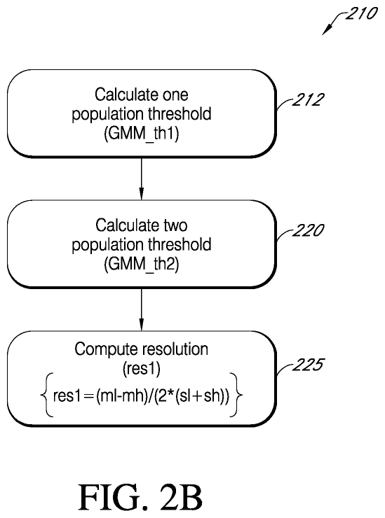

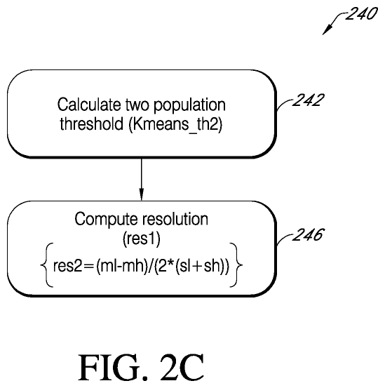

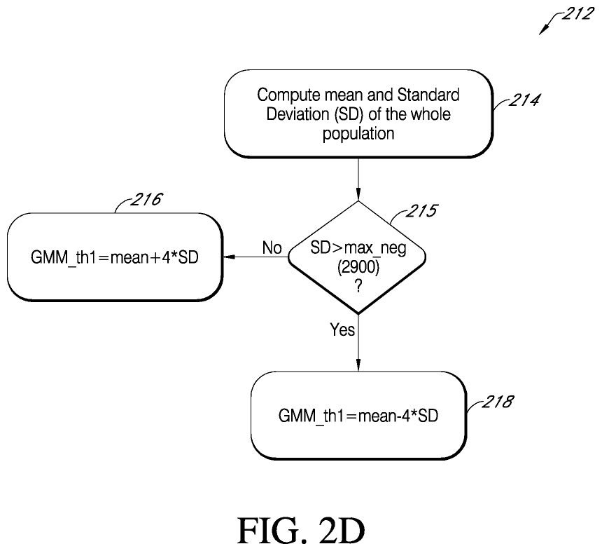

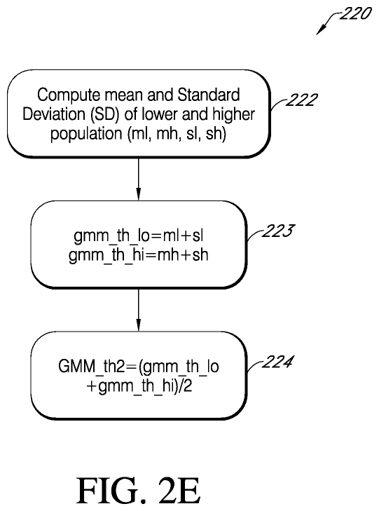

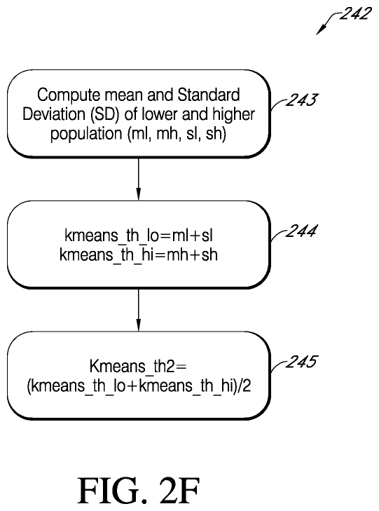

FIG. 2A is a flow diagram of a method for auto-thresholding of dPCR operative system of FIG. 1, in accordance with some embodiments. The auto-thresholding method 200 includes receiving fluorescent data corresponding to a plurality of optical signals detected at a dPCR instrument, wherein the fluorescent data includes a plurality of individual points associated with a plurality of microchambers in an action 202. The one or more clusters of the plurality individual points are data points that share a relative property. For example, the fluorescent data point of one cluster may indicate the presence of a portion of DNA or an infectious agent. That is, the shared relative property relates to the target population or the absence thereof. In an action 210, the auto-thresholding method 200 includes detecting whether the one or more clusters are two clusters associated with two distinct populations using Gaussian Mixture Modeling (GMM). For example, GMM engine 136 couples to the receiver 135 and the at least one memory 131 having instructions that cause the at least one processor 132 to detect whether the one or more clusters are two clusters associated with two distinct populations using GMM. In a decision action 230, method 200 includes detecting whether two distinct populations exist using GMM. In response to detecting two clusters using GMM, the auto-thresholding method 200 also includes assigning a two-population GMM threshold to be a final threshold in an action 235. In absence of detecting two distinct populations using GMM, the method 200 includes detecting whether the number of the one or more clusters is two clusters associated with a two-cluster population using K-means clustering in an action 240. For example, K-means clustering engine 137 in cooperation with processor 132 detects whether the number of the one or more clusters is two clusters associated with a two-cluster population using K-means clustering. In a decision action 250, method 200 includes detecting whether two distinct populations exist using K-means clustering. In response to detecting two clusters using K-means clustering, the auto-thresholding method 200 includes assigning a two-population K-means clustering threshold to be the final threshold in an action 255. In response to absence of detecting two clusters using K-means clustering, the auto-thresholding method 200 includes assigning a one-population GMM threshold to be the final threshold in an action 260.

FIG. 2B is a flow diagram of a method 210 for GMM fit as applied to the site statistics data of the auto-thresholding method 200 of FIG. 2A, in accordance with some embodiments. In an action 212, GMM fit method 210 includes calculating the one population threshold (GMM_th1). In an action 220, GMM fit method 210 includes calculating the two-population threshold (GMM_th2). In an action 225, GMM fit method 210 includes computing a first resolution (res1). In one example, the resolution (res1) equals the difference between the mean of GMM lower and GMM higher population divided by two times the sum of the standard deviation of the GMM lower and GMM higher population ((ml-mh)/(2*(sl+sh))).

FIG. 2C is a flow diagram of a method 240 for K-means clustering fit (K-means fit) as applied to the site statistics data of the auto-thresholding method 200 of FIG. 2A, in accordance with some embodiments. In an action 242, K-means clustering fit method 240 includes calculating the two-population threshold (Kmeans_th2). In an action 246, K-means clustering method 240 includes computing a second resolution (res2). In one example, the resolution (res2) equals the difference between the mean of K-means lower and K-means higher population divided by two times the sum of the standard deviation of K-means lower and K-means higher population ((ml-mh)/(2*(sl+sh))).

FIG. 2D is a flow diagram of a method 212 for calculating one population threshold using GMM of the auto-thresholding method of FIG. 2B, in accordance with some embodiments. In an action 214, method 212 includes computing the mean and Standard Deviation (SD) of the whole population. In a decision action 215, the method 212 includes detecting whether the standard deviation is greater than a maximum negative threshold. In an example, the standard deviation of the whole population derived using GMM must be greater than a maximum negative threshold of 2900.00, in order for the one population to be detected and an associated GMM threshold to be calculated. In response to the standard deviation being less than or equal to the maximum negative, the GMM threshold for one population is equal to the sum of the mean and four times the standard deviation (GMM_th1=mean±4*SD), in an action 216. In response to the standard deviation being greater than the maximum negative, the GMM threshold for one population is equal to the difference between the mean and four times the standard deviation (GMM_th1=mean−4*SD), in an action 218.

FIG. 2E is a flow diagram of method 220 for calculating two-population threshold using GMM of the auto-thresholding method 210 of FIG. 2B, in accordance with some embodiments. In action 222, GMM fit method 210 includes computing the mean and Standard Deviation (SD) of GMM lower and GMM higher population. In particular, variables associated with the GMM fit include mean of GMM lower population (ml), mean of GMM higher population (mh), standard deviation of lower population (sl), standard deviation of GMM higher population (sh), mean-mean for whole dataset, standard deviation for whole dataset (SD). In an action 223, GMM fit method 210 includes computing the GMM threshold for the GMM lower and GMM higher population. In particular, the GMM threshold for the lower population equals the sum of the mean of the lower population and the standard deviation for the lower population (gmm_th_lo=ml+sl); whereas, the GMM threshold for the GMM higher population equals the difference between the mean of the GMM higher population and the standard deviation for the GMMhigher population (gmm_th_hi-mh-sh). In an action 223, GMM fit method 210 includes computing the GMM threshold for a two-population response. For example, the GMM threshold for a two population equals the average of the GMM threshold for the GMM lower and GMM higher population (e.g. GMM_th2=(gmm_th_lo+gmm_th_hi)/2).

FIG. 2F is a flow diagram of a method 242 for calculating two population threshold using K-means clustering of the auto-thresholding method 240 of FIG. 2C, in accordance with some embodiments. In an action 243, K-means clustering fit method 240 includes computing the mean and Standard Deviation (SD) of K-means lower and K-means higher population (ml, mh, sl, sh). In an action 244, K-means clustering fit method 240 includes computing the K-means clustering threshold for the K-means lower and K-means higher population. In particular, the K-means threshold for the K-means lower population equals the sum of the mean of the K-means lower population and the standard deviation for the K-means lower population (means_th_lo=ml+sl); whereas, the K-means threshold for the K-means higher population equals the difference between the mean of the K-means higher population and the standard deviation for the K-means higher population (kmeans_th_hi=mh-sh). In an action 245, K-means clustering fit method 240 includes computing the K-means clustering threshold for a two-population response. For example, the K-means clustering threshold for a two population equals the average of the K-means clustering threshold for the K-means lower and K-means higher population (e.g., kmeans_th2=(kmeans_th_lo+kmeans_th_hi)/2).

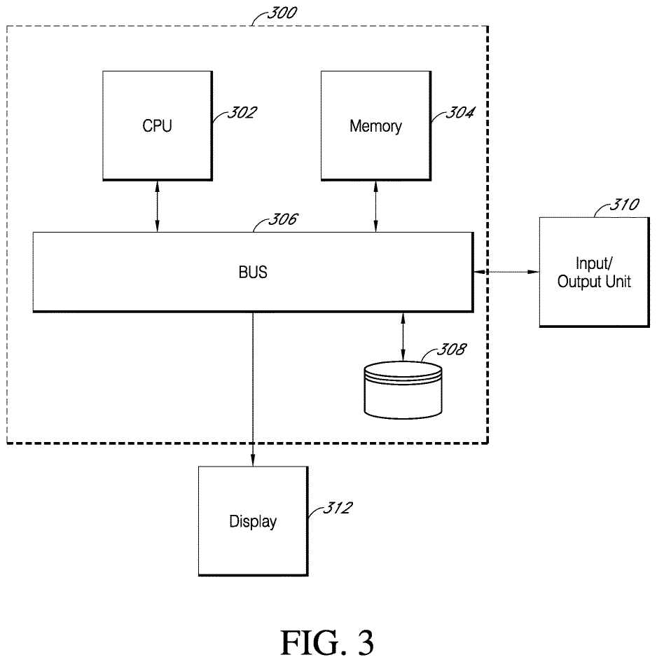

Referring now to FIG. 3, an illustration showing an example of a computing device which may implement the examples described herein is displayed. In the example, computer system 300 may provide one or more of the components of an dPCR/analysis configured to implement one or more logic modules and artificial neural networks and associated components for a computer-implemented dPCR analysis and associated interactive graphical user interface. It should be appreciated that the methods described herein may be performed with a digital processing system, such as a conventional, general-purpose computer system. Special purpose computers, which are designed or programmed to perform only one function may be used in the alternative. The computing device of FIG. 3 may be used to perform embodiments of the functionality for performing dPCR analysis in accordance with some embodiments. The computing device includes a central processing unit (CPU) 302, which is coupled through a bus 306 to a memory 304, and mass storage device 308. Mass storage device 308 represents a persistent data storage device such as a floppy disc drive or a fixed disc drive, which may be local or remote in some embodiments. The mass storage device 308 could implement a backup storage, in some embodiments. Memory 304 may include read only memory, random access memory, etc. Applications resident on the computing device may be stored on or accessed through a computer readable medium such as memory 304 or mass storage device 308 in some embodiments. Applications may also be in the form of modulated electronic signals modulated accessed through a network modem or other network interface of the computing device. It should be appreciated that CPU 302 may be embodied in a general-purpose processor, a special purpose processor, or a specially programmed logic device in some embodiments.

Display 312 is in communication with CPU 302, memory 304, and mass storage device 308, through bus 306. Display 312 is configured to display any visualization tools or reports associated with the system described herein. Input/output device 310 is coupled to bus 306 in order to communicate information in command selections to CPU 302. It should be appreciated that data to and from external devices may be communicated through the input/output device 310. CPU 302 can be defined to execute the functionality described herein to enable the functionality described with reference to FIGS. 1A-2F. The code embodying this functionality may be stored within memory 304 or mass storage device 308 for execution by a processor such as CPU 302 in some embodiments. The operating system on the computing device may be iOS™, MS-WINDOWS™, OS/2™, UNIX™, LINUX™, or other known operating systems. It should be appreciated that the embodiments described herein may be integrated with virtualized computing system also.

In the above description, numerous details are set forth. It will be apparent, however, to one skilled in the art, that the present invention may be practiced without these specific details. In some instances, well-known structures and devices are shown in block diagram form, rather than in detail, in order to avoid obscuring the present invention.

It is to be understood that the above description is intended to be illustrative, and not restrictive. Many other embodiments will be apparent to those of skill in the art upon reading and understanding the above description. Although the present invention has been described with reference to specific exemplary embodiments, it will be recognized that the invention is not limited to the embodiments described but can be practiced with modification and alteration within the spirit and scope of the appended claims. Accordingly, the specification and drawings are to be regarded in an illustrative sense rather than a restrictive sense. The scope of the invention should, therefore, be determined with reference to the appended claims, along with the full scope of equivalents to which such claims are entitled.

Detailed illustrative embodiments are disclosed herein. However, specific functional details disclosed herein are merely representative for purposes of describing embodiments. Embodiments may, however, be embodied in many alternate forms and should not be construed as limited to only the embodiments set forth herein.

It should be understood that although the terms first, second, etc. may be used herein to describe various steps or calculations, these steps or calculations should not be limited by these terms. These terms are only used to distinguish one step or calculation from another. For example, a first calculation could be termed a second calculation, and, similarly, a second step could be termed a first step, without departing from the scope of this disclosure. As used herein, the term “and/or” and the “I” symbol includes any and all combinations of one or more of the associated listed items. As used herein, the singular forms “a”, “an” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved. With the above embodiments in mind, it should be understood that the embodiments might employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing. Any of the operations described herein that form part of the embodiments are useful machine operations. The embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines can be used with computer programs written in accordance with the teachings herein, or it may be more convenient to construct a more specialized apparatus to perform the required operations.

A module, an application, a layer, an agent or other method-operable entity could be implemented as hardware, firmware, or a processor executing software, or combinations thereof. It should be appreciated that, where a software-based embodiment is disclosed herein, the software can be embodied in a physical machine such as a controller. For example, a controller could include a first module and a second module. A controller could be configured to perform various actions, e.g., of a method, an application, a layer or an agent.

The embodiments can also be embodied as computer readable code on a non-transitory computer readable medium. The computer readable medium is any data storage device that can store data, which can be thereafter read by a computer system. Examples of the computer readable medium include hard drives, network attached storage (NAS), read-only memory, random-access memory, CD-ROMs, CD-Rs, CD-RWs, magnetic tapes, flash memory devices, and other optical and non-optical data storage devices. The computer readable medium can also be distributed over a network coupled computer system so that the computer readable code is stored and executed in a distributed fashion. Embodiments described herein may be practiced with various computer system configurations including hand-held devices, tablets, microprocessor systems, microprocessor-based or programmable consumer electronics, minicomputers, mainframe computers and the like. The embodiments can also be practiced in distributed computing environments where tasks are performed by remote processing devices that are linked through a wire-based or wireless network.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

In various embodiments, one or more portions of the methods and mechanisms described herein may form part of a cloud-computing environment. In such embodiments, resources may be provided over the Internet as services according to one or more various models. Such models may include Infrastructure as a Service (IaaS), Platform as a Service (PaaS), and Software as a Service (Saas). In IaaS, computer infrastructure is delivered as a service. In such a case, the computing equipment is generally owned and operated by the service provider. In the PaaS model, software tools and underlying equipment used by developers to develop software solutions may be provided as a service and hosted by the service provider. SaaS typically includes a service provider licensing software as a service on demand. The service provider may host the software, or may deploy the software to a customer for a given period of time. Numerous combinations of the above models are possible and are contemplated.

Various units, circuits, or other components may be described or claimed as “configured to” or “operable to” perform a task or tasks. In such contexts, the phrase “configured to” is used to connote structure by indicating that the units/circuits/components include structure (e.g., circuitry) that performs the task or tasks during operation. As such, the unit/circuit/component can be said to be configured to perform the task even when the specified unit/circuit/component is not currently operational (e.g., is not on). The units/circuits/components used with the “configured to” language include hardware; for example, circuits, memory storing program instructions executable to implement the operation, etc. Reciting that a unit/circuit/component is “configured to” perform one or more tasks is expressly intended not to invoke 35 U.S.C. 112, sixth paragraph, for that unit/circuit/component. Additionally, “configured to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In the context of the specification, at least the following embodiments are described. Embodiment 1 is a method of processing digital Polymerase Chain Reaction (dPCR) data of a biological sample. The method comprising receiving fluorescent data corresponding to a plurality of optical signals detected by a dPCR instrument, wherein the fluorescent data correspond to fluorescent intensities of a plurality of sample partitions; determining whether the fluorescent data are associated with two distinct populations using a first mathematical model; and determining, in response to a result that the fluorescent data are not associated with two distinct populations using the first mathematical model, whether the fluorescent data are associated with two distinct populations using a second mathematical model. Embodiment 2 is the method of embodiment 1, further comprising assigning a two-population threshold determined by the first mathematical model as a final threshold in response to the fluorescent data being determined as associated with two distinct populations using the first mathematical model.

Embodiment 3 is the method of any of embodiments 1 and 2, further comprising assigning a two-population threshold determined by the second mathematical model as a final threshold, in response to the fluorescent data being determined as associated with two distinct populations using the second mathematical model. Embodiment 4 is the method of any of embodiments 1 to 3, further comprising assigning a one-population threshold determined by the first mathematical model to be a final threshold in response to the fluorescent data being determined as not associated with two distinct populations using the second mathematical model.

Embodiment 5 is the method of any of embodiments 1 to 4, wherein the first mathematical model includes Gaussian Mixture Model (GMM). Embodiment 6 is the method of embodiment 5, wherein the second mathematical model includes K-means clustering model. Embodiment 7 is the method of embodiment 6, wherein determining whether the fluorescent data are associated with two distinct populations using GMM comprises defining a GMM fit for a GMM higher population and a GMM lower population; calculating a first threshold associated with the first cluster based on the GMM fit of the GMM higher population and the GMM lower population; calculating a second threshold associated with the second cluster based on the GMM fit of the GMM higher population and the GMM lower population; determining a GMM resolution of the first cluster and second cluster; and identifying, in response to the GMM resolution being greater than a first predetermine variable, the fluorescent data as being associated with two distinct populations.

Embodiment 8 is the method of embodiment 7, wherein defining the GMM fit for the first cluster and the second cluster comprises calculating mean of the GMM higher population, mean of the GMM lower population, and mean of the whole population including entire plurality of partitions; calculating standard deviation of the whole population, standard deviation of the GMM higher population, and standard deviation of the GMM lower population; setting the GMM threshold associated with the GMM lower population to be the sum of the mean of the GMM lower population and the standard deviation of the GMM lower population; and setting the GMM threshold associated with the GMM higher population to be the difference between the mean of the GMM higher population and the standard deviation of the GMM higher population.

Embodiment 9 is the method of embodiment 8, wherein, in response to the fluorescent data being associated with two distinct populations based on GMM, setting the final threshold for the fluorescent data comprises calculating the sum of GMM threshold of the GMM higher population and the GMM threshold of the GMM lower population; and dividing the sum by two to generate a first average as the final threshold.

Embodiment 10 is the method of embodiment 9, wherein the final threshold, in response to the fluorescent data not being associated with two distinct populations based K-means clustering is obtained, using GMM, by the steps of detecting whether the difference of the mean of the whole population and the standard deviation of the whole population is greater than a second predetermined variable; setting, in response to detected difference greater than the second predetermined variable, the final threshold of the one-cluster population to be the difference between the mean of the whole population and four times the standard deviation of the whole population; and setting, in response to detected difference less than the predetermined variable, the final threshold of fluorescent data to be the sum between the mean of the whole population and four times the standard deviation of the whole population.

Embodiment 11 is the method of any of embodiments 6 to 10, wherein detecting whether the fluorescent data are associated with two distinct populations using K-means clustering comprises defining a K-means fit for a K-means higher population and a K-means lower population; calculating a first K-means threshold associated with the first cluster based on the K-means fit of the K-means higher population and the K-means lower population; calculating a second K-means threshold associated with the second cluster based on the K-means fit of the K-means higher population and the K-means lower population; determining the K-means resolution of the first cluster and second cluster; identifying, in response to the K-means resolution being greater than or equal to a third predetermine variable, the fluorescent data as being associated with two distinct populations; and identifying, in response to the K-means resolution is less than the third predetermine variable, the fluorescent data as being not associated with two distinct populations.

Embodiment 12 is the method of embodiment 11, wherein defining the K-means fit for the fluorescent data associated with two distinct populations comprises calculating mean of the K-means higher population, wherein the K-means higher population is a cluster that represents sample partitions with the presence of a target analyte; calculating mean of the K-means lower population, wherein the K-means lower population is a cluster representing sample partitions absent of the target analyte; calculating standard deviation of the K-means higher population and standard deviation of the K-means lower population; setting the K-means threshold of the K-means higher population to be the difference between the mean of the K-means higher population and the standard deviation of the K-means higher population; and setting the K-means threshold of the K-means lower population to be the sum of the mean of the K-means lower population and the standard deviation of the lower population.

Embodiment 13 is the method of embodiment 12, wherein, in response to the fluorescent data associated with two distinct populations based on K-means clustering, the final threshold for the fluorescent data is obtained by calculating the sum of K-means threshold of the K-means higher population and the K-means threshold of the K-means lower population; dividing the sum by two to generate a second average; and setting the K-means threshold associated with a two-cluster population to be the second average.

Embodiment 14 is the method of any of embodiments 11 to 13, wherein the K-means resolution is calculated as the absolute value of the difference between the mean of the K-means lower population and the mean of the K-means higher population, divided by two times the sum of the standard deviation of the K-means lower population and the standard deviation of the K-means higher population. Embodiment 14 is the method of any of embodiments 2 to 14, wherein data points of the fluorescent data above the final threshold correspond to sample partitions that contain a target analyte, and data points of the fluorescent data below the final threshold correspond to sample partitions without the target analyte. Embodiment 16 is the method of embodiment 15, wherein the target analyte includes a nucleic acid molecule, or a nucleic acids fragment. Embodiment 17 is the auto-thresholding method of any of embodiments 2-16, wherein the final threshold is configured to facilitate quantification of target analyte.

Embodiment 18 is a non-transitory computer-readable medium including instructions that is executable by one or more computer processors to perform the method of any one of claims 1-17.

Embodiment 19 is an auto-thresholding system for data obtained using an dPCR instrument, the auto-thresholding system comprising a memory; and a processor configured to receiving fluorescent data corresponding to a plurality of optical signals detected by a dPCR instrument, wherein the fluorescent data correspond to fluorescent intensities of a plurality of sample partitions; determining whether the fluorescent data are associated with two distinct populations using a first mathematical model including Gaussian Mixture Model (GMM); and determining, in response to the fluorescent data not being associated with two distinct populations using the first mathematical model, whether the fluorescent data are associated with two distinct populations using a second mathematical model including K-means clustering.

Embodiment 20 is the auto-thresholding system of embodiment 19, wherein determining whether the fluorescent data are associated with two distinct populations using the first mathematical model includes defining a GMM fit for a GMM higher population and a GMM lower population of the plurality of data points; calculating a first threshold associated with the GMM higher population based on the GMM higher population and the GMM lower population; calculating a second threshold associated with the GMM lower population based on the GMM higher population and the GMM lower population; determining the GMM resolution of the first and second cluster; and identifying in response to the GMM resolution being greater than a first predetermined variable, the plurality of data points as being associated with two distinct populations; and identifying in response to the GMM resolution being no greater than the first predetermined variable, the plurality of data points as not being associated with two distinct populations.

Embodiment 21 is the auto-thresholding system of embodiment 20, wherein the GMM resolution is calculated as the absolute value of the difference between the mean of the GMM lower population and the mean of the GMM higher population, divided by two times the sum of the standard deviation of the GMM lower population and the standard deviation of the GMM higher population. Embodiment 22 is the auto-thresholding system of any of embodiments 19 to 21, wherein determining whether the fluorescent data are associated with two distinct populations using the K-means clustering includes defining a K-means fit for a K-means higher population and a K-means lower population; calculating a first K-means threshold associated with the first cluster based on the K-means fit of the K-means higher population and the K-means lower population; calculating a second K-means threshold associated with the second cluster based on the K-means fit of the K-means higher population and the K-means lower population; determining the K-means resolution of the first cluster and second cluster; identifying, in response to the K-means resolution being greater than or equal to a third predetermine variable, the fluorescent data as being associated with two distinct populations; and identifying, in response to the K-means resolution is less than the third predetermine variable, the fluorescent data as being not associated with two distinct populations.

Embodiment 23 is the auto-thresholding system of embodiment 22, wherein the K-means resolution is calculated as the absolute value of the difference between the mean of the K-means lower population and the mean of the K-means higher population, divided by two times the sum of the standard deviation of the K-means lower population and the standard deviation of the K-means higher population. Embodiment 24 is the auto-thresholding system of any of claims 19 to 23, wherein the processor is further configured to assign a two-population threshold determined by GMM as a final threshold in response to the fluorescent data being determined as associated with two distinct populations using GMM. Embodiment 25 is the auto-thresholding system of claim 24, wherein the two-population threshold determined by GMM is obtained by dividing a sum of GMM threshold of the GMM higher population and the GMM threshold of the GMM lower population by 2.

Embodiment 26 is the auto-thresholding system of any of embodiments 19 to 25, wherein the processor is further configured to assign a two-population threshold determined by K-means clustering as a final threshold in response to the fluorescent data being determined as associated with two distinct populations using K-means clustering. Embodiments 27 is the auto-thresholding system of embodiment 23, wherein the two-population threshold determined by K-means clustering is obtained by dividing a sum of K-means threshold of the K-means higher population and the K-means threshold of the K-means lower population by 2. Embodiment 28 is the auto-thresholding system of any of embodiments 19 to 27, wherein the processor is further configured to assign a one-population threshold determined by GMM to be a final threshold in response to the fluorescent data being determined as not associated with two distinct populations using K-means clustering.

Embodiments 29 is the auto-thresholding system of embodiment 28, wherein the one-population threshold determined by GMM is obtained by detecting whether the difference of the mean of the whole population and the standard deviation of the whole population is greater than a second predetermined variable; setting, in response to the difference being greater than the second predetermined variable, the final threshold of the fluorescent data to be the difference between the mean of the whole population and four times the standard deviation of the whole population; and setting, in response to detected difference less than the predetermined variable, the final threshold of fluorescent data to be the sum between the mean of the whole population and four times the standard deviation of the whole population.

Embodiment 30 is the auto-thresholding system of any of embodiments 24 to 29, wherein data points of the fluorescent data above the final threshold correspond to sample partitions that contain a target analyte, and data points of the fluorescent data below the final threshold correspond to sample partitions without the target analyte. Embodiment 31 is the auto-thresholding system of embodiment 30, wherein the target analyte includes a nucleic acid molecule, or a nucleic acids fragment. Embodiment 32 is the auto-thresholding system of any of embodiments 24-31, wherein the final threshold is configured to facilitate quantification of target analyte. Embodiment 33 is a digital Polymerase Chain Reaction (dPCR) system, the system comprising a server, having at least one processor coupled to at least one memory and a dPCR processing module including an auto-thresholding system, wherein the auto-thresholding system comprises a cluster detection unit having a receiver coupled to the at least one memory having computer-readable instructions stored thereon that, when executed by the at least one processor cause the at least one processor to receive, from a dPCR instrument, fluorescent data corresponding to a plurality of optical signals detected by the dPCR instrument; wherein the fluorescent data includes a plurality of intensity values corresponds to a plurality of sample partitions; a Gaussian Mixture Modeling (GMM) engine coupled to the receiver and the at least one memory having computer-readable instructions stored thereon that, when executed by the at least one processor cause the at least one processor to detect whether the fluorescent data are two clusters associated with two distinct populations using Gaussian Mixture Modeling (GMM); assign, in response to detecting two clusters using GMM, a two-population GMM threshold to be a final threshold; a K-means clustering engine coupled to the GMM engine and the at least one memory having computer-readable instructions stored thereon that, when executed by the at least one processor cause the at least one processor to detect, in response to not detecting two distinct populations using GMM, whether the fluorescent data is two clusters associated with two distinct populations using K-means clustering; assign, in response to detecting two clusters using K-means clustering, a two-population K-means threshold to be the final threshold; and assign, in response to absence of detecting two clusters using K-means clustering, a one-population GMM threshold to be the final threshold.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

1. A method of processing digital Polymerase Chain Reaction (dPCR) data of a biological sample, the method comprising:
receiving fluorescent data corresponding to a plurality of optical signals detected by a dPCR instrument, wherein the fluorescent data correspond to fluorescent intensities of a plurality of sample partitions;
determining whether the fluorescent data are associated with two distinct populations using a first mathematical model; and
determining, in response to a result that the fluorescent data are not associated with two distinct populations using the first mathematical model, whether the fluorescent data are associated with two distinct populations using a second mathematical model.

2. The method of claim 1, further comprising assigning a two-population threshold determined by the first mathematical model as a final threshold in response to the fluorescent data being determined as associated with two distinct populations using the first mathematical model.

3. The method of claim 1, further comprising assigning a two-population threshold determined by the second mathematical model as a final threshold, in response to the fluorescent data being determined as associated with two distinct populations using the second mathematical model.

4. The method of claim 1, further comprising assigning a one-population threshold determined by the first mathematical model to be a final threshold in response to the fluorescent data being determined as not associated with two distinct populations using the second mathematical model.

5. The method of claim 1, wherein the first mathematical model includes Gaussian Mixture Model (GMM).

6. The method of claim 5, wherein the second mathematical model includes K-means clustering model.

7. The method of claim 6, wherein determining whether the fluorescent data are associated with two distinct populations using GMM comprises:
defining a GMM fit for a GMM higher population and a GMM lower population;
calculating a first threshold associated with the first cluster based on the GMM fit of the GMM higher population and the GMM lower population;
calculating a second threshold associated with the second cluster based on the GMM fit of the GMM higher population and the GMM lower population;
determining a GMM resolution of the first cluster and second cluster; and
identifying, in response to the GMM resolution being greater than a first predetermine variable, the fluorescent data as being associated with two distinct populations.

8. The method of claim 7, wherein defining the GMM fit for the first cluster and the second cluster comprises:
calculating mean of the GMM higher population, mean of the GMM lower population, and mean of the whole population including entire plurality of partitions;
calculating standard deviation of the whole population, standard deviation of the GMM higher population, and standard deviation of the GMM lower population;
setting the GMM threshold associated with the GMM lower population to be the sum of the mean of the GMM lower population and the standard deviation of the GMM lower population; and
setting the GMM threshold associated with the GMM higher population to be the difference between the mean of the GMM higher population and the standard deviation of the GMM higher population.

9. The method of claim 8, wherein, in response to the fluorescent data being associated with two distinct populations based on GMM, setting the final threshold for the fluorescent data comprises:
calculating the sum of GMM threshold of the higher population and the GMM threshold of the GMM lower population; and
dividing the sum by two to generate a first average as the final threshold.

10. The method of claim 9, wherein the final threshold, in response to the fluorescent data not being associated with two distinct populations based K-means clustering is obtained, using GMM, by the steps of:
detecting whether the difference of the mean of the whole population and the standard deviation of the whole population is greater than a second predetermined variable;
setting, in response to detected difference greater than the second predetermined variable, the final threshold of the one-cluster population to be the difference between the mean of the whole population and four times the standard deviation of the whole population; and
setting, in response to detected difference less than the predetermined variable, the final threshold of fluorescent data to be the sum between the mean of the whole population and four times the standard deviation of the whole population.

11. The method of any of claim 6, wherein detecting whether the fluorescent data are associated with two distinct populations using K-means clustering comprises,
defining a K-means fit for a K-means higher population and a K-means lower population;
calculating a first K-means threshold associated with the first cluster based on the K-means fit of the K-means higher population and the K-means lower population;
calculating a second K-means threshold associated with the second cluster based on the K-means fit of the K-means higher population and the K-means lower population;
determining the K-means resolution of the first cluster and second cluster;
identifying, in response to the K-means resolution being greater than or equal to a third predetermine variable, the fluorescent data as being associated with two distinct populations; and
identifying, in response to the K-means resolution is less than the third predetermine variable, the fluorescent data as being not associated with two distinct populations.

12. The method of claim 11, wherein defining the K-means fit for the fluorescent data associated with two distinct populations comprises:
calculating mean of the K-means higher population, wherein the K-means higher population is a cluster that represents sample partitions with the presence of a target analyte;
calculating mean of the K-means lower population, wherein the K-means lower population is a cluster representing partitions that is absent of the target analyte;
calculating standard deviation of the K-means higher population and standard deviation of the K-mean lower population;
setting the K-means threshold of the K-means higher population to be the difference between the mean of the K-means higher population and the standard deviation of the K-means higher population; and
setting the K-means threshold of the K-means lower population to be the sum of the mean of the K-means lower population and the standard deviation of the K-means lower population.

13. The method of claim 12, wherein, in response to the fluorescent data associated with two distinct populations based on K-means clustering, the final threshold for the fluorescent data is obtained by:
calculating the sum of K-means threshold of the K-means higher population and the K-means threshold of the K-means lower population;
dividing the sum by two to generate a second average; and
setting the K-means threshold associated with a two-cluster population to be the second average.

14. The method of claim 11, wherein the K-means resolution is calculated as the absolute value of the difference between the mean of the K-means lower population and the mean of the K-means higher population, divided by two times the sum of the standard deviation of the K-means lower population and the standard deviation of the K-means higher population.

15. The method of claim 2, wherein data points of the fluorescent data above the final threshold correspond to sample partitions that contain a target analyte, and data points of the fluorescent data below the final threshold correspond to sample partitions without the target analyte.

16. The method of claim 15, wherein the target analyte includes a nucleic acid molecule, or a nucleic acids fragment.

17. The method of claim 2, wherein the final threshold is configured to facilitate quantification of target analyte.

18. A non-transitory computer-readable medium including instructions that is executable by one or more computer processors to perform the method of claim 1.

19-33. (canceled)
